(19) United States
(12) Patent Application Publication
(10) Pub. No.: US 2020/0069437 A1
(43) Pub. Date: Mar. 5, 2020

(54) Graft Packing System for Medical Implants and Related Method

(71) Applicants: Thomas Zink, San Antonio, TX (US); Thomas Slott, Plano, TX (US); Steven Lee, Walnut Creek, CA (US); Frank Kuwamura, San Antonio, TX (US)

(72) Inventors: Thomas Zink, San Antonio, TX (US); Thomas Slott, Plano, TX (US); Steven Lee, Walnut Creek, CA (US); Frank Kuwamura, San Antonio, TX (US)

(21) Appl. No.: 16/559,720

(22) Filed: Sep. 4, 2019

Related U.S. Application Data
(60) Provisional application No. 62/727,263, filed on Sep. 5, 2018.

(51) Int. Cl. A61F 2/46; A61F 2/28; A61B 50/33; A61M 5/315

(52) U.S. Cl. CPC A61F 2/4601; A61F 2/28; A61B 50/33; A61F 2002/4625; A61F 2002/2835; A61F 2002/4649; A61F 2310/00005; A61M 5/31581

(57) ABSTRACT

A graft packing system comprised of a graft packing tray and insertion device and a method of using the same if disclosed. The graft packing tray is useful for storing and/or transporting a medical implant, wherein the tray is designed to permit the introduction of biological and/or bio-reactive material into the interior of the tray and the interior of the medical implant being stored and/or transported therein in a sterile and efficient manner via the insertion device. The graft packing tray may also be equipped with a reservoir containing pre-existing biological and/or bio-reactive material that can be introduced to the medical implant at the appropriate time via the insertion device.

### CROSS-REFERENCE TO RELATED APPLICATION

The present application claims priority from U.S. Provisional Patent Application Ser. No. 62/727,263 filed on Sep. 5, 2018, which is incorporated herein by reference.

### FIELD OF THE INVENTION

The present invention relates to a graft packing system for medical implants, and a related method of packing the medical implant with biological and/or bio-reactive materials in a sterile environment. More specifically, the invention relates to a graft packing tray for storing and/or transporting a medical implant, wherein the tray is designed to permit the introduction of biological and/or bio-reactive material into an interior cavity of the tray and the medical implant being stored and/or transported therein in a sterile and efficient manner. Further, the invention also discloses an insertion device for pulling and/or pushing the biological and/or bio-reactive material into the interior cavity of the tray and, more specifically, into the interior of the medical implant in the tray a precise and timely manner.

### BACKGROUND

Medical implants are well known in the medical arts for treating patients, particularly with respect to the musculoskeletal system. Historically, orthopedic implants, such as interbody fusion devices, osteotomy wedges and the like, have been made from machined titanium, polyether ether ketone (PEEK) and similar materials, and the design of said implant devices was based on the materials used with little, if any, consideration given to the internal structure of the implant device. As a result, such historic orthopedic implant devices have not always integrated well with the adjacent bone structure.

Notwithstanding, recent developments in three dimensional printing and additive manufacturing have allowed for more intricate designs of medical implant devices, and increased attention to the design of the interior portion of said medical devices. More specifically, titanium has a porosity that invites bone to integrate into the structure of the medical device, and recently developed three dimensional printing techniques permit the creation of more intricate interior spaces or cavities within the implant devices, such as titanium implants, into which biological and/or bio-reactive materials may be injected to enhance the osteo- or osseo-integration process between the medical implant and the adjacent bone structure of the patient.

Further, heretofore, biological and/or bio-reactive materials useful for promoting osteo-integration between an implant device and the surrounding bone structure had to be inserted or packed into the medical implant in the surgical field on the sterile table as part of the surgical procedure, which could be time consuming and could result in contamination because the packing occurs in an open air environment. More specifically, the medical implant is typically placed on a plate or in a block in an open air environment, and the biological and/or bio-reactive materials are forced into a large interior opening or hole in the medical implant device. The need for such a large central opening in the medical implant also limits design possibilities with respect to the medical implant device.

Consequently, there is a long felt need in the art for a graft packing tray and related insertion device for inserting biological and/or bio-reactive materials into the medical implant in a sterile environment. Further, there is also a long felt need in the art for a graft packing tray that does not require the removal of the implant from the tray in order to insert the biological and/or bio-reactive materials therein, thereby reducing the likelihood of contamination to the implant or the biological and/or bio-reactive materials during the insertion procedure (e.g., caused by inserting the biological and/or bio-reactive materials into the implant in an open air environment). There is also a long felt need in the art for a medical implant that does not require a large center opening or hole therein which, in turn, permits the use of more anatomical medical implant designs with less possibility of implant subsidence, increased contact surface between the implant device and the surrounding bone, and better patient outcomes. Finally, there is a long felt need in the art for a graft packing tray that is relatively easy to manufacture and that is safe and effective to use.

The present invention discloses a unique graft packing system comprised of a unique graft packing tray and related insertion device that provide a safe, efficient and cost effective manner of inserting biological and/or bio-reactive materials into a medical implant in a sterile environment, thereby reducing the likelihood of contamination and promoting osteo-integration between the medical implant and the surrounding bone. The unique graft packing tray and related insertion device also permit both the medical implant and the biological and/or bio-reactive materials to be stored and transported in one device, but separate and apart until necessary.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

In one embodiment, the graft packing tray of the present invention comprises a housing having an interior cavity contained therein for storing and/or transporting a medical implant, such as an interbody fusion device, osteotomy wedge or the like, and one or more ports in fluid communication with said cavity for the delivery of a biological and/or bio-reactive material to said medical implant via said one or more ports and the insertion device and in a sterile environment. In a preferred embodiment of the present invention, the medical implant will have at least one mesh surface or subsurface to further promote the osteo-integration of the implant device to the patient's bone, and the medical implant will also have an interior space and/or void for receiving the biological and/or bio-reactive material.

The graft packing system of the present invention preferably further comprises a syringe-like insertion device for injecting the biological and/or bio-reactive material through one or more of said ports in the graft packing tray and into both the interior cavity of the packing tray and the medical implant positioned therein. The insertion device is preferably comprised of a handle, a plunger that travels within an interior cavity of the insertion device, and a nozzle. More specifically, the biological and/or bio-reactive material may be placed into the cavity of the insertion device and forced through the nozzle and into the packing tray and/or medical implant by urging the handle and the related plunger of the insertion device in the direction of the nozzle.

In a further embodiment of the present invention, the graft packing tray of the present invention may further comprise an interior reservoir for housing or storing the biological and/or bio-reactive material until needed. More specifically, the reservoir containing the biological and/or bio-reactive material may be positioned between, and in fluid communication with, one of said ports and the interior cavity that houses the medical implant device. The insertion device may then be used to inject air, a saline solution or other inert material through said port and into the reservoir, thereby forcing the biological and/or bio-reactive material contained in the reservoir to travel into the interior cavity housing the medical implant and the medical implant itself, particularly the interior space of the medical implant.

The graft packing tray and related insertion device of the present invention, in all of its potential embodiments, provides a safe, efficient and cost effective manner of inserting biological and/or bio-reactive materials into a medical implant in a sterile environment, thereby reducing the likelihood of contamination and promoting osteo-integration between the medical implant and the surrounding bone. Further, the graft packing tray of the present invention does not require the removal of the implant from the tray in order to insert the biological and/or bio-reactive materials therein, and the medical implant does not require a large center opening or hole therein which, in turn, permits the use of more anatomical medical implant designs with less possibility of implant subsidence, increased contact surface between the medical implant device and the surrounding bone, and better patient outcomes.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

While the specification concludes with claims particularly pointing out and distinctly claiming the present invention, it is believed that the present invention will be better understood from the following description in conjunction with the accompanying FIGS., in which like reference numerals identify like elements, and wherein:

FIG. 1 illustrates a partial perspective view of one embodiment of the graft packing tray of the present invention engaged with an insertion device in accordance with the disclosed architecture.

FIG. 2 illustrates a perspective view of implant mesh in accordance with the disclosed architecture.

FIG. 3 illustrates a top partial perspective view of one embodiment of the graft packing tray of the present invention in accordance with the disclosed architecture.

FIG. 4 illustrates a top perspective view of one embodiment of the graft packing tray of the present invention engaged with an insertion device in accordance with the disclosed architecture.

FIG. 5 illustrates a front partial perspective view of one embodiment of the graft packing tray of the present invention engaged with an insertion device in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

Generally stated, the present invention relates to a unique graft packing system comprised of a graft packing tray and related insertion device for inserting biological and/or bio-reactive materials into a medical implant in a sterile environment, thereby reducing the likelihood of contamination and promoting osteo-integration between the medical implant and the surrounding bone once implanted. Further, the graft packing tray may be used to store and/or transport the medical implant until needed for implantation.

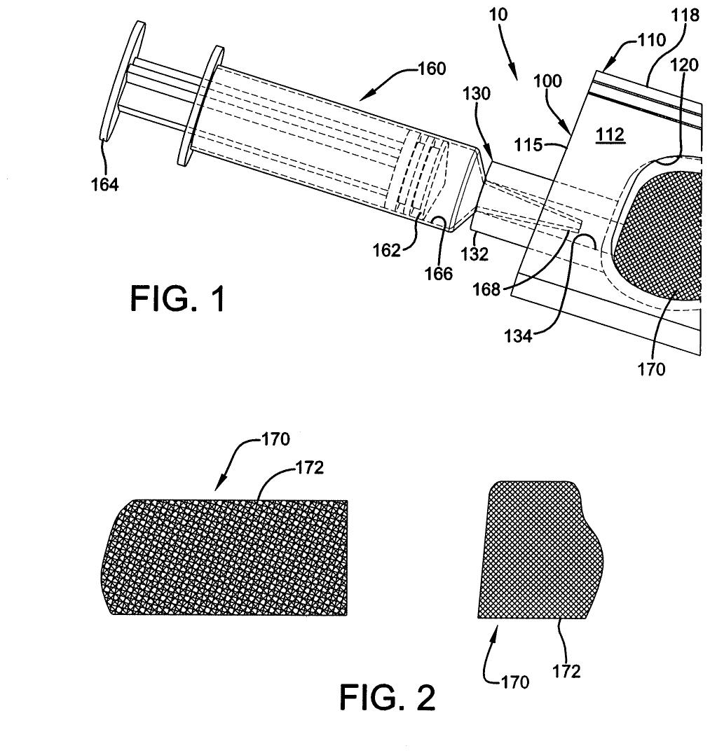

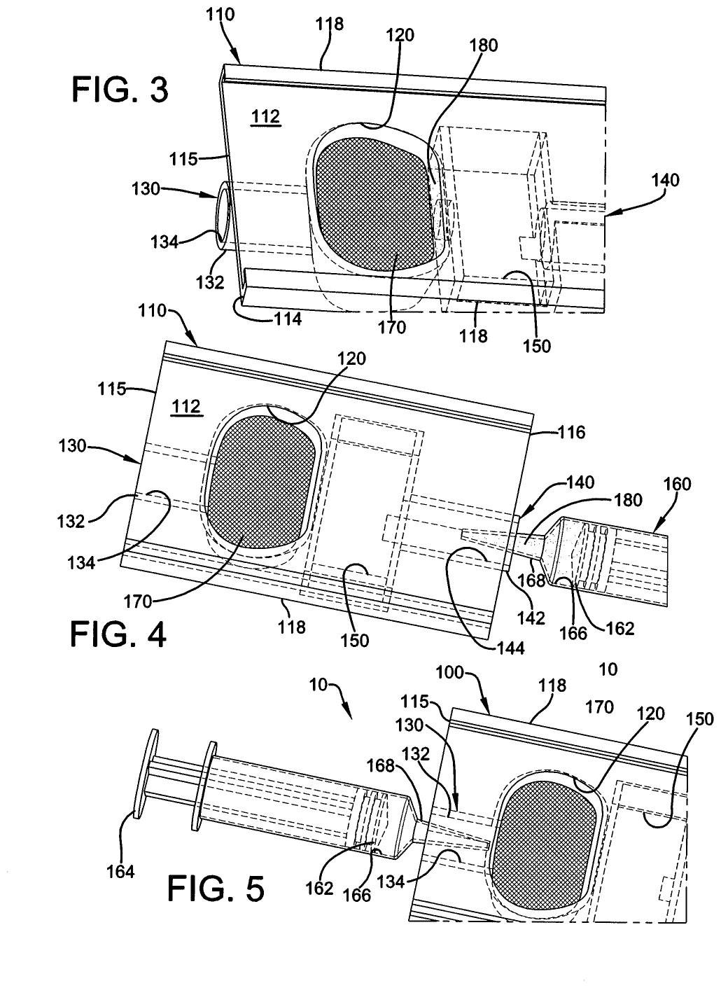

Referring initially to the drawings, FIG. 1 illustrates a partial perspective view of one embodiment of the graft packing system 10 of the present invention, which is preferably comprised of a graft packing tray 100 and an insertion device 160. In a preferred embodiment of the present invention, graft packing tray 100 is generally rectangular in shape and is comprised of a housing 110, an interior cavity 120, a first port 130, and a second port 140. Notwithstanding, one of ordinary skill in the art will appreciate that the shape and size of the graft packing tray 100, as shown in FIGS. 1, 3, 4 and 5, are for illustrative purposes only, and that many other shapes and sizes of graft packing tray 100 are well within the scope of the present disclosure. Although the shape and particular dimensions of the graft packing tray 100 (i.e., length, width, and height) are important design parameters for good performance, the graft packing tray 100 may be any shape or size that ensures optimal performance during use and is within the overall objective of the present invention.

Housing 110 is also preferably generally rectangular in shape and is further comprised of a top 112, a bottom 114, a first end 115, a second end 116 and opposing sides 118, though it is contemplated that other shapes could be used as well without affecting the overall concept of the present invention. Housing 110 is preferably comprised of polyethylene, or other suitable durable and medical grade material for maintaining a sterile environment, and reducing the possibility of contamination.

Interior cavity 120 is positioned within housing 110 between top 112, bottom 114, first end 115, second end 116 and opposing sides 118, and can be any shape or size necessary to house, store and/or transport a medical implant 170. Interior cavity 120 is in fluid communication with a first port 130 and/or a second port 140 for enabling interior cavity 120 and the medical implant 170 stored therein to be filled with a biological and/or bio-reactive material 180, as described more fully below.

First port 130 may be a continuous opening extending from said first end 115 to interior cavity 120 as shown in FIG. 5, or it may be a generally cylindrical (or other shape) structure 132 with a continuous opening 134 therein that extends from the outer end of structure 132 to interior cavity 120, as best shown in FIG. 1. Similarly, second port 140 may be a continuous opening extending from said second end 116 to interior cavity 120, or it may be a generally cylindrical (or other shape) structure 142 with a continuous opening 144 therein that extends from the outer end of structure 142 to interior cavity 120.

Alternatively, graft packing tray 100 may further comprise a reservoir 150 for storing biological and/or bio-reactive material 180 that may be in fluid communication with, and positioned between, both interior cavity 120 and second port 140, as described more fully below. Reservoir 150 may be any shape or size necessary to house, store and/or transport enough biological and/or bio-reactive material 180 to completely fill interior cavity 120 and medical implant 170. In this manner, both medical implant 170 and biological and/or bio-reactive material 180 can be stored in one convenient and sterile location, albeit separate and apart until it becomes necessary to combine the same, for example, in anticipation of a surgical procedure.

Medical implant 170 may be any medical implant known in the art and is preferably comprised of a titanium alloy, though it is contemplated that other medical grade materials can also be used without affecting the overall concept of the present invention. Medical implant 170 is preferably manufactured using additive manufacturing techniques, such as three dimensional printing, to enable more intricate designs and increased focus on the internal structure of medical implant 170 to better promote osteo-integration between the implant 170 and the adjacent bone structure of the patient. For example, medical implant 170 may have one or more mesh surfaces or subsurfaces 172 for promoting bone growth therein, as best illustrated in FIG. 2. Nonetheless, other manufacturing techniques for manufacturing medical implant 170 are also contemplated, such as casting, molding, forming, etc. Medical implant 170 may be sterilized by any currently known method of sterilization before placing the same in cavity 120 of graft packing tray 100 to further reduce the possibility of contamination.

As previously discussed, medical implant 170 is positioned within the interior cavity of 120 of graft packing tray 100 for storage and/or transportation until needed for implantation. Nonetheless, as described more fully below, a user (not shown) can inject biological and/or bio-reactive material 180 to fill interior cavity 120 and medical implant 170 via the first port 130, the second port 140, and/or the reservoir 150 using insertion device 160.

More specifically, insertion device 160 is a syringe like device that can be used to inject biological and/or bio-reactive material 180 into the interior cavity 120 and medical implant 170 via the first port 130, the second port 140, and/or the reservoir 150 to promote osteo-integration between the medical implant 170 and the surrounding bone (not shown) following implantation. It has been determined that the inclusion of one or more mesh surfaces or subsurfaces 172 in medical implant 170 is particularly effective for promoting osteo-integration between the implant and the surrounding bone post procedure.

Insertion device 160 is preferably comprised of a plunger 162, a handle 164, a cavity 166, and a nozzle 168. More specifically, similar to a syringe, a material, such as biological and/or bio-reactive material 180, may be placed within cavity 166 and then expelled from insertion device 160 through nozzle 168 by using the handle 164 to advance the plunger 162 through the cavity 166 in the direction of nozzle 168. Likewise, insertion device 160 can also function as a vacuum to pull a material, such as biological and/or bio-reactive material 180, in the direction of insertion device 160 when the handle 164 is used to pull the plunger 162 through the cavity 166 in a direction that is opposite, or away from, nozzle 168, as explained more fully below. Notwithstanding, it is also contemplated that other types of insertion devices, such as mechanical pumps, syringes, pneumatic pumps and the like, could also be used without negatively impacting the overall concept of the present invention.

Biological and/or bio-reactive material 180 may be any material currently used to sterile pack implants, and that promotes osteo-integration between a medical implant 170, particularly an implant with a mesh surface or subsurface 172, and the patient's surrounding bone structure. Another important aspect of the present invention is the user's ability to know the exact volume of biological and/or bio-reactive packing material 180 needed in advance of the procedure to insure that the entire medical implant 170 is properly packed with the biological material 180 to substantially eliminate any voids in the implant device 170 or its mesh structure 172.

Having described a preferred embodiment of the graft packing system 10, a preferred method of its use will now be described in general terms. A user (not shown) desiring to prepare a medical implant 170 contained in the cavity 120 of graft packing tray 100 for implantation into a patient could insert a biological and/or bio-reactive material 180 within cavity 166 of insertion device 160 and insert the nozzle 168 of said device 160 into the first port 130 or second port 140 of graft packing tray 100, as best shown in FIGS. 1 and 4, respectively. Once inserted, the user can then use the handle 164 to advance the plunger 162 through the cavity 166 in the direction of the nozzle 168, thereby discharging the biological and/or bio-reactive material 180 through said nozzle 168, through said first or second ports 130, 140, and into cavity 120 and medical implant 170 in a sterile environment. Once both cavity 120 and medical implant 170 are filled with the biological and/or bio-reactive material 180, the insertion device 160 may be removed from the graft packing tray 100, and implant 170 (packed with the biological and/or bio-reactive material 180) may be stored and/or transported until needed for implantation into a patient.

Alternatively, if graft packing tray 100 further comprises the reservoir 150 between first or second ports 130, 140 and interior cavity 120, biological and/or bio-reactive material 180 may be preloaded and stored into said reservoir 150 and, at the appropriate time, insertion device 160 could be filled with air, a saline solution, or other inert substance and administered through nozzle 168, through second port 140, and into reservoir 150 to force the biological and/or bio-reactive material 180 from reservoir 150 and into interior cavity 120 and medical implant device 170.

As a further alternative, if graft packing tray 100 further comprises the reservoir 150 between first or second ports 130, 140 and interior cavity 120, biological and/or bio-reactive material 180 may be preloaded and stored into said reservoir 150 and, at the appropriate time, pulled (as opposed to being pushed) into interior cavity 120 and medical implant 170 by connecting insertion device 160 to the port 130, 140 opposite of reservoir 150, such that medical implant 170 and interior cavity 120 are positioned there between, and using insertion device 160 as a vacuum to pull the biological and/or bio-reactive material 180 from the reservoir 150 and into both interior cavity 120 and medical implant 170, as best shown in FIG. 5. More specifically, the handle 164 and plunger 162 can be pulled in a direction opposite nozzle 168, thereby creating a suction or vacuuming force to pull the biological and/or bio-reactive material 180 in the direction of insertion device 160 and into both interior cavity 120 and medical implant device 170.

Accordingly, the unique graft packing system 10 of the present invention, in each of its various possible embodiments, provides a safe, efficient and cost effective manner of inserting biological and/or bio-reactive materials into a medical implant in a sterile environment, thereby reducing the likelihood of contamination and promoting osteo-integration between the medical implant and the surrounding bone structure once implanted. Further, the graft packing tray of the present invention does not require the removal of the implant from the tray in order to insert the biological and/or bio-reactive materials therein, and the medical implant does not require a large center opening or hole therein which, in turn, permits the use of more anatomical medical implant designs with less possibility of implant subsidence, increased contact surface between the medical implant device and the surrounding bone, and better patient outcomes.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A graft packing system comprising:
a graft packing device; and
an insertion device.

2. The graft packing system of claim 1, wherein said graft packing device further comprises a housing, a cavity inside of said housing, and a first port in communication with both said cavity and an exterior of the housing.

3. The graft packing system of claim 2 further comprising a second port.

4. The graft packing system of claim 3 further comprising a reservoir in communication with both said second port and said cavity.

5. The graft packing system of claim 1, wherein the insertion device further comprises a plunger, a cavity and a nozzle.

6. The graft packing system of claim 2 further comprising a medical implant and a biological packing material.

7. The graft packing system of claim 6, wherein a portion of said biological packing material is positioned within the medical implant and the medical implant is positioned within the cavity.

8. The graft packing system of claim 4, wherein a biological packing material is contained in the reservoir.

9. The graft packing system of claim 1, wherein a biological packing material is introduced to the graft packing device via the insertion device.

10. A graft packing device for storing a medical implant and a biological packing material, wherein the graft packing device comprises:
a housing having a cavity therein; and
a first port in communication with both said cavity and an exterior of the housing.

11. The graft packing device of claim 10 further comprising a reservoir and a second port.

12. The graft packing device of claim 11, wherein the second port is in communication with both the reservoir and the exterior of the housing, and further wherein the reservoir is in further communication with the cavity.

13. The graft packing device of claim 11, wherein the medical implant is contained in the cavity and the biological packing material is contained in the reservoir.

14. The graft packing device of claim 13, wherein an insertion device is used to move the biological packing material from the reservoir and into both the cavity and the medical implant.

15. The graft packing device of claim 10, wherein the medical implant has at least one mesh surface or mesh subsurface.

16. The graft packing device of claim 10, wherein an insertion device is used to insert the biological packing material into both the cavity and the medical implant via the first port.

17. A method of preparing a medical implant with a biological packing material in a sterile environment comprising the steps of:
placing the medical implant into a cavity in a graft packing device comprised of a housing and a first port, wherein the first port is in communication with both the cavity and an exterior of the housing;
inserting the biological packing material into an insertion device having a nozzle;
inserting the nozzle of the insertion device into the first port; and
injecting the biological packing material into the cavity and the medical implant via the nozzle.

18. The method of claim 17, wherein the graft packing device further comprises a reservoir in communication with both a second port and the cavity.

19. The method of claim 18, wherein the biological packing material is in the reservoir and is advanced from the reservoir to the cavity and the medical implant via the insertion device via the second port.

20. The method of claim 19, wherein the insertion device discharges air or a saline solution into the reservoir via the nozzle and the second port to advance the biological packing material into the cavity and the medical implant.
